(19) United States
(12) Patent Application Publication
(10) Pub. No.: US 2026/0263752 A1
(43) Pub. Date: Sep. 10, 2026

(54) SELF-RETAINING CATHETERS AND METHODS OF USE

(71) Applicant: THE CHILDREN'S MEDICAL CENTER CORPORATION, Boston, MA (US)

(72) Inventor: Michael P. Kurtz, Brookline, MA (US)

(73) Assignee: THE CHILDREN'S MEDICAL CENTER CORPORATION, Boston, MA (US)

(21) Appl. No.: 19/480,002

(22) PCT Filed: May 1, 2024
(86) PCT No.: PCT/US2024/027148

§ 371 Date: Oct. 29, 2025

Related U.S. Application Data
(60) Provisional application No. 63/499,916, filed on May 3, 2023.

(51) Int. Cl. A61M 25/04; A61M 25/00; A61M 25/01; A61M 25/02

(52) U.S. Cl. CPC A61M 25/04; A61M 25/0017; A61M 25/0052; A61M 25/0105; A61M 2025/0286; A61M 2210/1085

(57) ABSTRACT

Self-retaining catheter systems are generally described. A catheter system may reduce the risk of premature dislodgement of the catheter from a patient's anatomy (e.g., lumen, organ, natural or surgical opening). In some embodiments, a catheter system may include a tether extending from a portion proximate to the proximal end of a tubular body of the system to a distal portion. Applying tension to the tether may transition the tubular body from a linear configuration to a looped or coiled configuration, sized to be larger than an opening of the patient's lumen or organ. The expanded configuration of the tubular body may reduce the risk of premature dislodgement. In some embodiments, the catheter system may include a reinforced portion coupled to a proximal end of the tether to help reinforce the tubular body in its looped configuration. In some embodiments, the catheter system may be a pediatric urinary catheter.

### FIELD

Disclosed embodiments are related to catheter systems and related methods. More specifically, systems and methods for self-retaining catheter systems are disclosed.

### BACKGROUND

Catheter systems are conventionally used to facilitate fluid flow into or out of an anatomical cavity. Catheters are typically tubular bodies which are inserted into a patient's cavity, lumen, or orifice to fluidically connect the anatomy with an external fluid source (e.g., for flushing) or a fluid container (e.g., for drainage). The catheter can be placed in a natural or surgically formed opening. Urinary drainage catheters in particular are typically inserted into a patient's bladder through the urethral tract to facilitate urine drainage for patients who cannot naturally express urine, possibly following a surgical procedure, incontinence, orthopedic incapacitation.

### SUMMARY

In some embodiments, catheter systems are disclosed. The catheter system may include a tubular body, the tubular body having a proximal end configured to be arranged in a bladder of a patient, a reinforced portion proximal to and offset from the proximal end of the tubular body, and a tether coupled to the reinforced portion, the tether extending from the reinforced portion to a distal end of the tubular body, wherein at least a portion of the tether is disposed inside the tubular body.

In some embodiments, methods of operating catheter systems are disclosed. The method includes grasping a distal end of a tether coupled to a reinforced portion of the system, the reinforced portion disposed proximate to a proximal end of a tubular body, and transitioning the tubular body from a relaxed configuration to a looped configuration. It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1C show a catheter system according to some embodiments;

FIGS. 2A-2B show an exemplary catheter system in both relaxed and looped configurations; and

FIGS. 3A-3C show three exemplary catheter systems arranged in a patient's urinary system.

### DETAILED DESCRIPTION

Flexible urinary catheters are typically placed in a patient's urethral canal to help drain urine from a patient's bladder into a urine bag or diaper. Catheters are typically employed when the patient is unable to naturally and controllably express urine. For example, male pediatric patients who have undergone a surgical procedure following a hypospadias diagnosis might require a urinary catheter to facilitate urine expression for some time after their procedure. As such, a urinary catheter can be placed through the infant's urethra and into the bladder, fluidically connecting the urine-filled bladder with a diaper or collection bag external to the infant's urethral opening.

The inventors have recognized that conventional urinary catheters exhibit risks of being prematurely dislodged from a patient's urethra and bladder due to their linear nature. In other words, catheters can prematurely slide out of a patient's urethra due to movement or perturbations, requiring a clinician to reinsert the catheter, which can be a timely and uncomfortable process. In particular, catheters inserted into small pediatric patients exhibit higher risks of premature dislodgement due to the more active nature of pediatric patients (e.g., kicking legs). The activity level of pediatric patients also presents challenges for clinicians during the insertion process of the catheter. Thus, the inventors have recognized a need to reduce the risks of premature dislodgement and reduce the potential for repetitive catheter insertion.

In adult patients, balloon catheters can be used to limit the risks of dislodgement. Such catheters (e.g., Foley catheters) include an inflatable bladder. Such a catheter is inserted into a patient's lumen (e.g., urethra) and once a portion of the catheter has passed the patient's bladder, the inflatable bladder of the catheter can be inflated through a fluid source (e.g., air) to increase the cross-sectional area of the catheter relative to the urethra, and prevent premature dislodgement. As is known, to remove the catheter, the balloon is deflated, reducing its overall footprint, and thereby allowing the catheter to be withdrawn from the urethra. Alternatives to balloon catheters, including catheters which structurally expand without fluid input can also be used. However, the inventors have recognized that expandable catheters typically exhibit cross-sectional areas with tolerances incompatible with the small anatomies of pediatric patients.

In some instances, pediatric catheters are sutured directly to the patient anatomy to reduce the risk of dislodgement. Given the smaller length scales of pediatric patients, as well as their active nature, placing and also removing sutures following treatment can pose significant challenges to clinicians. Highly skilled clinicians are typically needed to carefully apply and/or remove the sutures to reduce the risk of damaging the patient's urological system, which may have recently undergone a surgical procedure, while managing the patient's severe discomfort and activity.

In view of the foregoing, the inventors have recognized a need for a catheter system to reduce the risk of premature dislodgement for various patient anatomical sizes, particularly, pediatric patients. The catheter system may have an insertion profile or cross-sectional area comparable to conventional catheters while exhibiting self-retaining properties. The catheter system may remain securely within the patient anatomy without the use of sutures, and may be readily removed when the catheter system is no longer required. However, instances in which different benefits are offered by the systems and methods disclosed herein are also possible.

In some embodiments, a catheter system may include a generally tubular body having a proximal end configured to be positioned in a patient's bladder or other desired organ, and a distal end configured to remain external to the patient for drainage or flushing purposes. The tubular body may be flexible. The tubular body may be formed as a straight tube or may have a suitable curvature along all or parts of its length. The tubular body may facilitate fluid flow into and/or out of the patient's organ (and/or any other surgical or natural opening in the patient anatomy). For example, the catheter system may be a urinary catheter to help drain urine from a patient's bladder.

In some embodiments, the catheter system may include a stiffener arranged proximate to the proximal end of the tubular body. The stiffener, which may be a body arranged within the tubular body, may be coupled to a flexible tether extending from the stiffener to the distal end of the body. The tether may pass from the stiffener, through an outlet port formed in the body, back into the tubular sidewalls of the body through an inlet port, and along the tubular sidewalls to the distal end of the body. The tether may include a handling portion at its distal end, positioned external to the distal end of the tubular body.

In some embodiments, the handling portion may be grasped to tension the tether and help transition the tubular body from its relaxed linear configuration into a looped or coiled configuration, following placement within the patient's lumen/organ. The looped configuration may help retain the catheter system within the patient anatomy and reduce the risk of premature dislodgement. In some embodiments, the looped configuration may be formed akin to a pigtail catheter. In contrast to conventional pigtail catheters, the stiffener of the catheter systems described herein may reinforce the tubular body against the anatomy (e.g., bladder neck) to further reduce the risk of the catheter system being pulled out of the anatomy unexpectedly. In some embodiments, the stiffener may reduce the risk of the tether pulling through the tubular sidewall material.

In some embodiments, the catheter system may include a cuff removably coupled to the distal end of the tubular body. The cuff may be configured to help retain the tether in its tensioned state while the catheter system is inserted into the patient anatomy. In some embodiments, the cuff may limit the axial displacement of the tether relative to the tubular sidewalls. When a clinician determines that the catheter system may be removed, the cuff may be readily removed from the distal end of the tubular body, allowing the tether, and subsequently the sidewalls of the tubular body, to relax back into their linear configuration, such that the system may be readily removed from the patient. The insertion, retention, and removal process of the catheter systems described herein may be facilitated through the tensioning and relaxing of a tether, and may therefore be conducted in a rapid and efficient manner without the use of sutures.

In some embodiments, the stiffener may be a separate body arranged within the tubular body of the catheter system. The stiffener may have a generally cylindrical shape and help reinforce the catheter system in its looped configuration. As will be described in greater detail below, in some embodiments, the stiffener may be formed integrally with the tubular body. It should be appreciated that although in some embodiments, the stiffener may be formed of a more mechanically stiff material to reinforce the tubular sidewalls, embodiments in which the stiffener is formed of the same or similar material as the sidewalls are also contemplated. The additional material of the stiffener may help reinforce the sidewalls despite the properties of the stiffener itself. In some embodiments, a stiffener formed of a less mechanically stiff material than the sidewalls may be employed.

It should be appreciated that the tether may be flexible, such that it may be configured in a variety of different shapes, but may not exhibit significant in-line elasticity. The limited elasticity of the tether may allow the tether to reconfigure the catheter system prior to extending along its own axial direction, which may improve the efficiency of the system. In other words, the applying tension to the tether may rearrange the configuration of the catheter system, rather than stretch the tether. The tether may be formed of a sufficiently flexible material such that it may weave through one or more ports of the body and include a graspable handling end. In some embodiments, the tether may have minimal in-line elasticity, such that grasping the handling end of the tether to apply tension to the tether facilitates the transition of the catheter system from the relaxed configuration to its looped configuration rather than axially elongating the tether. In some embodiments, the tether may be formed of conventional suture materials, although other embodiments are also contemplated. In some embodiments, the tether may be formed of a surgical suture material. The tethers described herein may be any suitable generally flexible, one-dimensional structure formed from an arrangement and/or assembly of yarns, fibers, strings, strands, threads, monofilaments, and/or multifilaments. Such structures may comprise knits, weaves, braids, non-woven materials, combinations thereof, and/or any other suitable form factor. The tether may be formed of any suitable biocompatible material or combination of materials, bioabsorbable and/or non-bioabsorbable. In some embodiments, the tether may be a suture. The suture may have a standard size defined by the United States Pharmacopeia (U.S.P.), including, but not limited to, 11-0, 10-0, 9-0, 8-0, 7-0, 6-0, 5-0, 4-0, 3-0, 2-0, 0, 1, 2, 3, 4, 5, 6, 7. The tether may be formed of any suitable material, including, but not limited to, polypropylene, nylon, silk, vicryl, monocryl, polyglactin, combinations thereof, and/or any other suitable conventional suture material.

The tubular sidewalls and/or the stiffener may be formed of any suitable biocompatible material. In some embodiments, the tubular sidewalls and/or the stiffener may be flexible to allow the catheter system to transition between the relaxed configuration and the looped or coiled configuration. The tubular sidewalls and/or the stiffener may be formed of a material compatible with bodily fluids (e.g., urine) for prolonged periods of time (e.g., days, weeks). In some embodiments, the tubular sidewalls and/or the stiffener may be formed of a material which exhibits limited biodegradability within the time period that the catheter system is installed in the patient's lumen. The tubular sidewalls and/or the stiffener of the catheter systems described herein may be formed entirely or partially of polytetrafluorethylene, silicones (e.g., platinum cured silicones, peroxide cured silicones), silastic materials, nylons, polygalactin, polyethylene (e.g., high density polyethylene), polyamide, polyvinylchloride, polyurethane, polyethyleneterephthalate, polypropylene, or copolymers, polyacrylonitrile, polyvinylacetates, polylacticacids, polyhydroxyalkanoates, polyesters, polycaprolactone, polyvinyl alcohol, polystyrene, polyethers, polycarbonates, polyamides, poly(acrylic acid), polyvinylchloride, polyacrylate, polyolefins, polystyrene, rubbers, polyethylene terephthalate, polyorthoester, polyanhydride, polyether sulfone, combinations thereof, and/or any other suitable material. It should be appreciated that tubular sidewalls and/or the stiffener may include any suitable coating or treatment to improve the comfort of the catheter system for the patient and/or improve fluid flow function of the system. In some embodiments, the tubular sidewalls and/or the stiffener may be formed of a composite material. In some embodiments, the tubular sidewalls and/or the stiffener may be partially formed of a nitinol material. Any suitable catheter material known in the art may be employed to form the tubular sidewalls and/or the stiffener, as the present disclosure is not so limited.

The catheter systems described herein may be employed for any suitable application. For example, in some embodiments, the catheter systems described herein may be employed to facilitate urine output from pediatric patients. The catheter systems may also be used for fluid flow into and/or out of a patient's organ or lumen. Some non-limiting exemplary embodiments of the catheter systems include, urethral catheters, ureteric catheters, catheters, suction catheters, pulmonary catheters, vaginal catheters, peritoneal catheters, chest tubes, pericardial drainage tubes, CNS drainage catheters or shunts, venous catheters, arterial catheters, peritoneal catheters, nasogastric tubes, feeding tubes, total parenteral nutrition tubes, gastric tubes, subcutaneous catheters, percutaneous catheters, esophageal catheters, biliary catheters, tracheal catheters, bronchial catheters, arterial catheters, combinations thereof, and/or any other suitable catheter type.

The catheter systems described herein may be placed in any suitable part of patient, including, but not limited to, the urethra, the kidneys, the bladder, the prostate, the uterus, the skin, the heart, the lungs, various organs in the abdomen, and/or any suitable portion of the patient. The catheter systems may be employed in pediatric and/or adult patients, as the present disclosure is not so limited.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

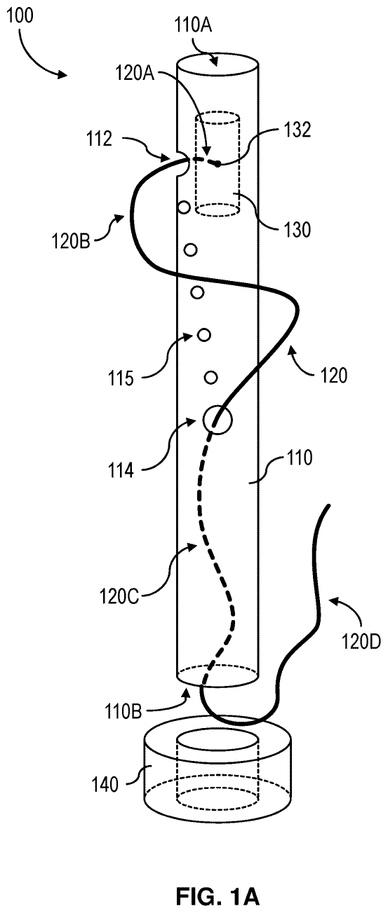

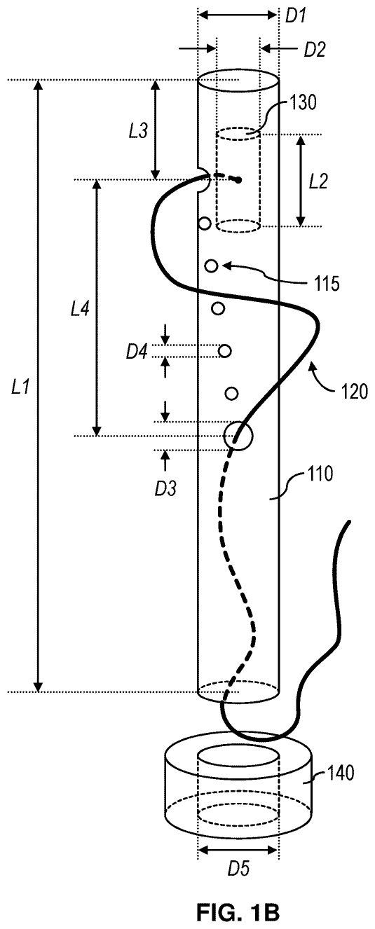

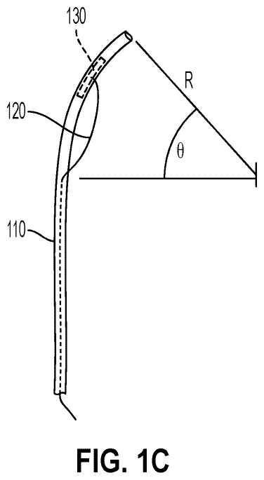

FIGS. 1A-1C depict a catheter system 100 according to some embodiments, which may be inserted partially into a patient's naturally formed lumen, such as a urethra.

The catheter system may include a tubular body 110 having sidewalls with a proximal end 110A and a distal end 110B. The tubular body may be formed of a flexible, bio-compatible material. In some embodiments, the body 110 may be formed of a material which may not be significantly degraded or otherwise affected by bodily fluids (e.g., urine, blood) and temperatures while the catheter remains inserted in the patient's lumen.

As shown in FIG. 1A, the body 110 of the catheter system 100 may have a general linear or cylindrical, yet flexible configuration. As described previously, while this linear configuration may facilitate the insertion of a catheter into the patient's lumen, the catheter may be accidentally dislodged from the lumen. Thus, the catheter systems of the present disclosure may include a tether 120 which may facilitate the reconfiguration of the body 110 and reduce the risks of the catheter dislodging from the patient's lumen. In some embodiments, the catheter systems of the present disclosure may be retained in place without the use of sutures.

The tether 120 may extend between a stiffener 130 positioned within the body 110 of the catheter system and the distal end 110B of the body. In some embodiments, as shown in FIG. 1A, the tether 120 may include a first portion 120A extending between a fixation point 132 on the stiffener 130 and an outlet port 112 formed on the body 110, a second portion 120B extending between the outlet port 112 and an inlet port 114 formed on the body 110, a third portion 120C extending between the inlet port 114 and the distal end 110B of the body 110, and a handling portion 120D extending out of the distal end 110B of the body. As indicated by contrasting solid and dashed lines, the first portion 120A and third portion 120C of the tether 120 may be arranged inside the sidewalls of the body 110, while the second portion 120B and the handling portion 120D may be arranged outside of the sidewalls. The tether 120 may be fixed to the stiffener in any suitable fashion, including, but not limited to a welding process, adhesives, knotting, and/or any other suitable method.

It should be appreciated that although four portions are shown and described, other arrangements of the tether relative to the catheter sidewalls are contemplated. The tether may include more or less than four portions, each of which may reside within or outside of the sidewalls of the body 110, as the present disclosure is not so limited.

In some embodiments, the tether 120 may be formed of flexible, but substantially inelastic material, such that applying tension to the handling portion 120D may result in the shortening of the various portions of the tether relative to the handling portion 120D. This effect may be facilitated by the fixation point 132 of the first portion 120A of the tether. Pulling on the handling portion of the tether may urge other portions of the tether, such as the first, second, and third, towards the distal end 110B of the body. Due to the fixation of the tether to the stiffener 130, as well as its arrangement relative to the outlet portion 112, the tether may modify the arrangement of the catheter body 110 between the outlet port 112 and the inlet port 114. In some embodiments, the catheter body may be transitioned into a looped or coiled configuration, akin to a pigtail, through tensioning of the tether. This looped configuration may increase the effective volumetric footprint of the catheter system and reduce the likelihood that the system may be prematurely dislodged from the lumen. For example, if the catheter system is inserted into a urethra, such that the proximal end 110A is arranged at least partially in the bladder, the size and arrangement of the catheter in its looped configuration may reduce the risk of the catheter falling back out through the urethra.

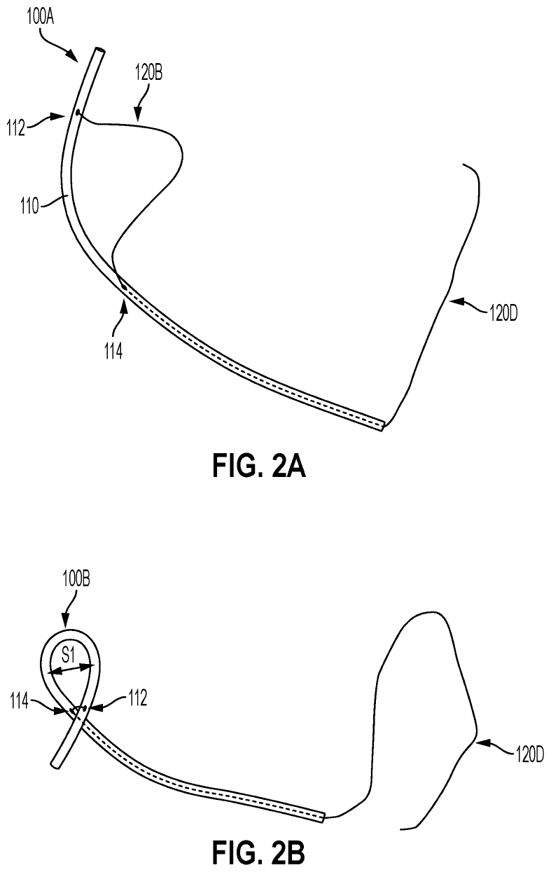

FIG. 2A shows an exemplary catheter system in a relaxed configuration 100A, with the catheter body 110 being in a generally elongated state, which may allow a clinician to insert the catheter into a patient's lumen (e.g., urethra). As shown in FIG. 2A, a portion 120B of the tether may extend external to the sidewalls of the body 110 between the outlet port 112 and the inlet port 114 of the catheter system. The system may also include a handling portion 120D at the distal end of the body. FIG. 2B shows the catheter system of FIG. 2A transitioned into a looped configuration 100B through tensioning of the handling portion 120D of the tether, as indicated by the length difference between the handling portion 120D shown in FIG. 2A compared to FIG. 2B. The tensioning of the tether may approximate the inlet 114 and outlet 112 ports, such that a portion of the body arranged in between the two ports may be urged into a curved loop. As described previously, in some embodiments, this process may be enabled by the fixation of the tether to a stiffener arranged internal to the sidewalls, close to the proximal end of the sidewalls. In some embodiments, the larger footprint of the loop formed by the body may reduce the risk of premature dislodgement of the catheter from the patient's lumen. embodiments where the catheter system transitions into a looped configuration with multiple loops are also contemplated.

Referring back to FIG. 1A, in some embodiments, the outlet port 112 through which the tether 120 extends may be arranged at different circumferential positions. For example, the outlet port 112 may be arranged at a first circumferential position relative to the axial direction of the tubular body, and the inlet port 114 may be arranged at a second circumferential position relative to the axial direction of the tubular body. In some embodiments, as represented by FIG. 1A, the two circumferential positions may be different from one another. For example, the two circumferential positions may be perpendicular to one another. It should be appreciated that any suitable arrangement between the inlet and outlet ports are contemplated.

Such an arrangement may reduce the risk of the tubular body from colliding with one another during the transition to the looped configuration. However, it should be appreciated that the sidewalls of the tubular body may be sufficiently flexible to overcome the collision and continue on to the fully looped configuration. In some embodiments, the sidewalls may be able to readily twist to accommodate the tensioning tether. Accordingly, embodiments in which the inlet and outlet ports are arranged at the same circumferential positions are also contemplated.

In some embodiments, the catheter system 100 may include one or more drainage holes 115 to allow fluid transport into and/or out of the patient's lumen. For example, in some embodiments, the drainage holes 115 may be used to drain urine out of a patient's bladder when the catheter is inserted at least partially into a patient's urethra. The one or more drainage holes 115 may be arranged in any suitable arrangement between the inlet 114 and outlet 112 ports, which may correspond to the portion of the body that has transitioned to the looped configuration. In this way, the drainage holes may be arranged about the loop of the catheter system in its looped configuration. The arrangement of drainage holes about the loop may facilitate consistent and efficient fluid flow through the ports.

In some embodiments, the drainage holes may be arranged at different circumferential positions relative to the axial direction of the tubular sidewalls. In other embodiments, the drainage holes may be arranged at the same circumferential position relative to the axial direction of the tubular sidewalls.

It should be appreciated that drainage holes arranged between the outlet port 112 and the proximal end 110A of the sidewalls are also contemplated, in combination with, or instead of the drainage holes shown in FIG. 1A. Similarly, drainage holes arranged between the inlet port 114 and the distal end of the sidewalls 110B are also contemplated, in combination with, or instead of the drainage holes described herein.

As described previously, the tether of FIG. 1A may be fixed to a stiffener 130 arranged within the tubular sidewalls 110. In some embodiments, a proximal end of the tether 120 may be fixed to a central portion of the stiffener at a fixation point 132. The arrangement of the fixation point 132 of the tether 120 relative to the stiffener 130 may allow the stiffener to abut against the opening of the patient's lumen (e.g., bladder neck) when the catheter system is in a looped configuration. As will be described in greater detail below, the stiffener 130 may accordingly reinforce the tubular sidewalls to further reduce the risk of catheter dislodgement from the patient's lumen. In some embodiments, the coupling of the tether to the stiffener relative to the tubular sidewalls may also reduce the risk of the tether tearing through the tubular sidewalls when tensioned.

In some embodiments, as represented by FIG. 1A, the stiffener 130 may be formed of a body arranged coaxially within the tubular sidewalls, proximal to the proximal end 110A of the catheter system. In the relaxed state of the catheter system, the stiffener may be freely moveable relative to the tubular sidewalls, but may be urged to a predetermined position when the tether is tensioned through its handling portion. In other embodiments, the stiffener 130 may be fixed (e.g., through a welding process, adhesives, press-fitting, etc.) to the tubular sidewalls, such that its axial position relative to the tubular sidewalls may not significantly be altered through the tensioning of the tether. In other embodiments still, the stiffener may be formed as part of the sidewalls, rather than a separate body. For example, in some embodiments, a portion of the catheter corresponding to the stiffener may not be hollow. In other embodiments, a portion of the catheter system corresponding to the stiffener may exhibit thicker sidewalls than other portions of the catheter system. Any suitable arrangement of the stiffener relative to the sidewalls to reinforce a portion of the sidewalls proximate to the proximal end of the tether is contemplated, as the present disclosure is not so limited.

In some embodiments, the catheter system 100 may include a cuff 140 to help retain the tether 120 in its tensioned state. As shown in FIG. 1A, the cuff 140 may be slid on to the distal end 110B of the tubular body and retain the tether axially in place relative to the sidewalls. In some embodiments, the cuff may limit or lock the axial displacement of the tether relative to the body. The cuff may therefore reduce the risk of the catheter system undesirably transitioning back into its relaxed state from the looped state. In some embodiments, when the catheter system is no longer necessary, the cuff may be removed from the catheter system 100 to allow the catheter to transition back into its relaxed state and be readily removed from the patient's lumen. In some embodiments, the distal end of the catheter may be cut or otherwise severed to help transition the catheter back into its relaxed state. In other embodiments, the cuff may be removable from the distal end of the catheter, such that upon removal of the cuff, the tether may transition back into its relaxed state to facilitate removal of the catheter system.

Alternative methods of cuff removal or disengagement are also contemplated. The cuff may be sized to slide axially around the distal end of the sidewalls while the handling portion is arranged on the outer surface of the body. Accordingly, the cuff 140 may couple the handling portion of the tether with the sidewalls and reduce the risk of the tether relaxing and transitioning the system back into its relaxed state.

It should be appreciated that although a ring-shaped cuff 140 is shown in FIG. 1A, the present disclosure is not limited to a ring-like structure, and alternative structures for limiting the axial translation of the tether relative to the body. It should also be appreciated that in some embodiments, the distal end of the body may include a coupling, such as a Luer-lock, to place the catheter system in fluid communication with an external volume and/or fluid source. For example, the catheter system may be irrigated by a fluid source through a connection at the distal end of the body. The body may include any suitable connective structure to allow fluid flow into and/or out of the patient, as the present disclosure is not so limited.

As shown in FIG. 1B, in some embodiments, the tubular body 110 of the catheter system may be characterized by an outer diameter D1. The outer diameter D1 may be any suitable size to fit within (e.g., smaller than) a patient's lumen. For example, the outer diameter D1 may be sized to fit within an infant urethral canal. The outer diameter D1 of the body 110 may be 2.3 mm. In some embodiments, the outer diameter D1 of the body 110 may be greater than or equal to approximately 1 mm, 1.5 mm, 2 mm, 2.3 mm, 2.5 mm, 3 mm, 3.5 mm, 4 mm,

4.5 mm, 5 mm, 5.5 mm, 6 mm, 7 mm, 8 mm, and/or any other suitable diameter. The outer diameter D1 of the body 110 may be less than or equal to approximately 8 mm, 7 mm, 6 mm, 5.5. mm, 5 mm, 4.5 mm, 4 mm, 3.5 mm, 3 mm, 2.5 mm, 2.3 mm, 2 mm, 1.5 mm, 1 mm, and/or any other suitable diameter. Combinations of the foregoing, including outer diameter D1 between 1 mm and 8 mm, are also contemplated. It should be appreciated that the body may have any suitable outer diameter, greater than and/or above the aforementioned ranges, as the present disclosure is not so limited.

In some embodiments, the tubular body 110 of the catheter system may be characterized by a length L1, as indicated in FIG. 1B. The length L1 may be any suitable size to extend at least partially within a patient's lumen. For example, the length L1 of the tubular body may be sufficiently long to extend along the entirety of an infant urethral canal, with its proximal end positioned in the patient's bladder and its distal end positioned external to the patient's urethra. In some embodiments, the length L1 of the tubular body may be approximately 26 cm. In some embodiments, the body length L1 may be greater than or equal to approximately 5 cm, 10 cm, 15 cm, 20 cm, 25 cm, 30 cm, 35 cm, 40 cm, and/or any other suitable length. The body length L1 may be less than or equal to approximately 40 cm, 35, 30 cm, 25 cm, 20 cm, 15 cm, 10 cm, 5 cm, and/or any other suitable length. Combinations of the foregoing, including body lengths between approximately 5 cm and 40 cm, between approximately 15 cm and 30 cm, are also contemplated. It should be appreciated that the body may have any suitable length, greater than and/or above the aforementioned ranges, as the present disclosure is not so limited.

As shown in FIG. 1B, the stiffener 130 may be characterized by a maximum diameter D2. In some embodiments, the stiffener diameter D2 may be determined by the size of the tubular sidewalls. For example, the stiffener diameter may be less than or approximately equal to the inner diameter of the tubular sidewalls. The stiffener diameter D2 may be any suitable proportion of the tubular body diameter D1, including, but not limited to, greater than or equal to approximately 25%, 30%, 40%, 50%, 60%, 70%, 80%, 90%, 95%, 98%, and/or less than or equal to approximately 98%, 95%, 90%, 80%, 70%, 60%, 50%, 40%, 30%, 25%, and/or any other suitable percentage of the tubular body diameter D1. Combinations of the foregoing, including stiffener diameters being between approximately 20% to 98% of the tubular body diameter are also contemplated. It should be appreciated that the stiffener may have any suitable diameter, greater than and/or above the aforementioned ranges relative to the tubular body diameter, as the present disclosure is not so limited.

The stiffener 130 may also be characterized by an axial length L2 to facilitate the reinforcement of the tubular sidewalls when the catheter system is in the looped configuration. It should be appreciated that the stiffener may have an axial length L2 sufficiently long to reduce the risk of the stiffener falling out of the outlet port. The axial length L2 of the stiffener may also be larger than an opening of the natural lumen, to reduce the risk of the catheter system unexpectedly falling out of the patient. The axial length L2 of the stiffener may also be smaller than a maximum width of an organ in which the catheter system is looped. For example, when the catheter system is inserted into a bladder through a urethral canal, the stiffener may be smaller than the width of the bladder to reduce the risk of discomfort.

In some embodiments, the stiffener length L2 may be greater than or equal to approximately 1 cm, 2 cm, 3 cm, 4 cm, 5 cm, 6 cm, 10 cm, and/or any other suitable length. For example, the stiffener length L2 may be approximately 3 cm. The stiffener length L2 may be less than or equal to approximately 10 cm, 6 cm, 5 cm, 4 cm, 3 cm, 2 cm, 1 cm, and/or any other suitable length. Combinations of the foregoing, including stiffener lengths between approximately 1 cm and 10 cm are also contemplated. It should be appreciated that the stiffener may have any suitable length, greater than and/or above the aforementioned ranges, as the present disclosure is not so limited.

It should be appreciated that although the stiffener is shown to have a generally cylindrical shape in FIGS. 1A-1C, alternative shapes such as ellipsoidal, tubular, polyhedral, helical, combinations thereof, and/or any other suitable shape. As described previously, in some embodiments, the stiffener may be formed integrally with the sidewalls.

As shown in FIGS. 1A-1C, the stiffener 130 may be arranged offset from the proximal end 110A of the tubular body. The central portion of the stiffener 130 may therefore be spaced a length L3 away from the outlet port 112. In instances where a proximal portion of the stiffener is positioned at the proximal end of the tubular body, the length L3 may correspond to half the length L2 of the stiffener. In other instances, the length L3 may be larger than half the length L2. The length L3 between the central portion of the stiffener and the proximal end of the tubular body 110 may be any suitable length to help reinforce the catheter system against premature or accidental dislodgement in its looped configuration. For example, the length L3 may be approximately 3 cm. The length L3 between the central portion of the stiffener and the proximal end of the tubular body 110 may be greater than or equal to approximately 1 cm, 2 cm, 3 cm, 4 cm, 5 cm, 6 cm, 8 cm, and/or any other suitable length. The length L3 may also be less than or equal to approximately 8 cm, 6 cm, 5 cm, 4 cm, 3 cm, 2 cm, 1 cm, and/or any other suitable length. Combinations of the foregoing, including between 1 cm and 8 cm are also contemplated. It should be appreciated that the length L3 may be any suitable length, greater than and/or above the aforementioned ranges, as the present disclosure is not so limited.

It should be appreciated that although the tether 120 is shown to be fixed to the stiffener at its central portion, embodiments in which the tether is fixed to other non-central portions of the stiffener are also contemplated.

As described previously, the catheter system may be transitioned between its relaxed configuration and a looped configuration by applying tension to a tether 120 fixed to a stiffener 130. When the tether 120 is tensioned, the length of the second portion 120B of the tether (see FIG. 1A) may be minimized as the length of the third portion 120C and handling portion 120D are increased to compensate for the reduced length of the second portion in order to bring the inlet and outlet ports together, while maintaining approximately the same total length of the tether. As the. The size of the loop formed in the looped configuration (see size S1 of loop shown in FIG. 2B) may be determined by the spacing between the outlet 112 and inlet 114 ports. The size of the loop may determine the efficiency of fluid flow into and/or out of the patient's organ (e.g., bladder). The distance L4 between the inlet 114 and outlet 112 ports may be any suitable distance to facilitate a desirable loop size depending upon the application of the catheter system. In embodiments where the system is employed in pediatric urethral applications, the distance L4 may be 9 cm. The distance L4 may be any suitable distance, greater than or equal to approximately 3 cm, 4 cm, 5 cm, 6 cm, 7 cm, 8 cm, 9 cm, 10 cm, 12 cm, 15 cm, 18 cm, 20 cm, 22 cm, 25 cm, 30 cm, and/or any other suitable distance. The distance L4 may also be less than or equal to approximately 30 cm, 25 cm, 22 cm, 20 cm, 18 cm, 15 cm, 12 cm, 10 cm, 9 cm, 8 cm, 7 cm, 6 cm, 5 cm, 4 cm, 3 cm, and/or any other suitable distance. Combinations of the foregoing, including distance LA between 3 cm and 30 cm are also contemplated. It should be appreciated that the length LA may be any suitable length, greater than and/or above the aforementioned ranges, as the present disclosure is not so limited.

Each of the inlet 114 and outlet 112 ports may have an effective diameter D3, as shown in FIGS. 1A-1B. The port diameter may be large enough to allow the tether 120 to pass through the ports with limited friction. Accordingly, in some embodiments, the port diameter D3 may be larger than the tether diameter. In some embodiments, the inlet and outlet ports may have the same diameter. In other embodiments, the inlet and outlet ports may be differently sized. Either of the inlet and outlet ports may be any suitable diameter D3, including, but not limited to, greater than or equal to approximately 0.5 mm, 1 mm, 2 mm, 3 mm, 5 mm, and/or less than or equal to approximately 5 mm, 3 mm, 2 mm, 1 mm, 0.5 mm, and/or any other suitable size.

Combinations of the foregoing, including inlet and/or outlet ports between approximately 0.5 mm and 5 are also contemplated. It should be appreciated that either of the inlet 114 and outlet 112 ports may have any suitable diameter D3, greater than and/or above the aforementioned ranges, as the present disclosure is not so limited.

In some embodiments, the catheter system 100 may include one or more drainage holes 115 for fluid flow into and/or out of the tubular body. The drainage holes 115 may have any suitable diameter D4, as shown in FIG. 1B, to enable controlled fluid flow through the ports. The drainage hole diameter D4 may be any suitable size, including greater than or equal to approximately 0.2 mm, 0.5 mm, 1 mm, 2 mm, 3 mm, 5 mm, and/or less than or equal to

approximately 5 mm, 3 mm, 2 mm, 1 mm, 0.5 mm, 0.2 mm, and/or any other suitable size.

Combinations of the foregoing are also contemplated, including drainage hole diameters D4 between approximately 0.2 and 5 mm. It should be appreciated that the drainage hole diameter D4 may be any suitable size, greater than and/or above the aforementioned ranges, as the present disclosure is not so limited. In some embodiments, all drainage holes may be sized similarly, whereas in other embodiments, the drainage holes may be differently sized.

As described previously, a cuff 140 may be used to help maintain the catheter system in its looped configuration. The cuff may reduce the risk of the tether 120 relaxing and undesirably dislodging the system from the patient's lumen. The cuff 140 may have an inner diameter D5, as shown in FIG. 1B, which may fit around the outer diameter of the tubular sidewalls 110. The cuff diameter D5 may be any suitable size to slide around the outer diameter of the tubular sidewalls. It should be appreciated that the cuff inner diameter D5 may be close enough to the outer diameter of the tubular sidewalls to reduce the risk of the tether slipping at the interface of the cuff and the sidewalls. In one exemplary embodiment, the outer diameter of the tubular sidewalls may be approximately 2.5 mm and the inner diameter D5 of the cuff 140 may be approximately 3 mm. The cuff diameter D5 may be any suitable size, including, but not limited to, greater than or equal to approximately 2 mm, 2.5 mm, 3 mm, 4 mm, 5 mm, 6 mm, 8 mm, 10 mm, less than or equal to approximately 10 mm, 8 mm, 6 mm, 5 mm, 4 mm, 3 mm, 2.5 mm, 2 mm, and/or any other suitable size. Combinations of the foregoing are also contemplated, including cuff inner diameter D5 between approximately 2 mm and 10 mm. It should be appreciated that the cuff inner diameter D5 may be any suitable size, greater than and/or above the aforementioned

As shown in FIGS. 1A-1B, the tether 120 may extend from a fixation point 132 on the stiffener 130 to a handling portion 120D at its distal end. The tether may have a length longer than the length L1 of the tubular body 110 to allow the handling portion 120D to rest outside of the distal end 110B of the body when the catheter system is in its relaxed state, allowing a clinician to grasp and apply tension to the tether. In some embodiments, the tether may have a length greater than or equal to approximately 8 cm, 10 cm, 15 cm, 20 cm, 25 cm, 30 cm, 40 cm,

50 cm, 60 cm, less than or equal to approximately 60 cm, 50 cm, 40 cm, 30 cm, 25 cm, 20 cm, 15 cm, 10 cm, 8 cm, and/or any other suitable length. Combinations of the foregoing, including tether lengths between 8 cm and 60 are also contemplated, as well as tether lengths shorter than or longer than the aforementioned ranges.

In one embodiment, the tubular body may be formed such that it includes a slight curvature to encourage the tubular body to transition into the looped or coiled configuration following placement within the patient's lumen/organ. In one embodiment, the insertion end (e.g., at the proximal end) of the tubular body may be preformed into a curved shape. In one embodiment, the curved shape is suitable to help transition into the looped or coiled configuration yet not so curved so as to exert excessive pressure or force on the urethra upon insertion and/or withdrawal. As shown in FIG. 1C, insertion end of the tubular body may be formed with a radius of curvature R extending over an angle of @. In one embodiment, the radius of curvature R is approximately 10 centimeters. In one embodiment, the angle @ is approximately 30 degrees. Other suitable radii and angles may be employed as the present disclosure is not limited in this respect.

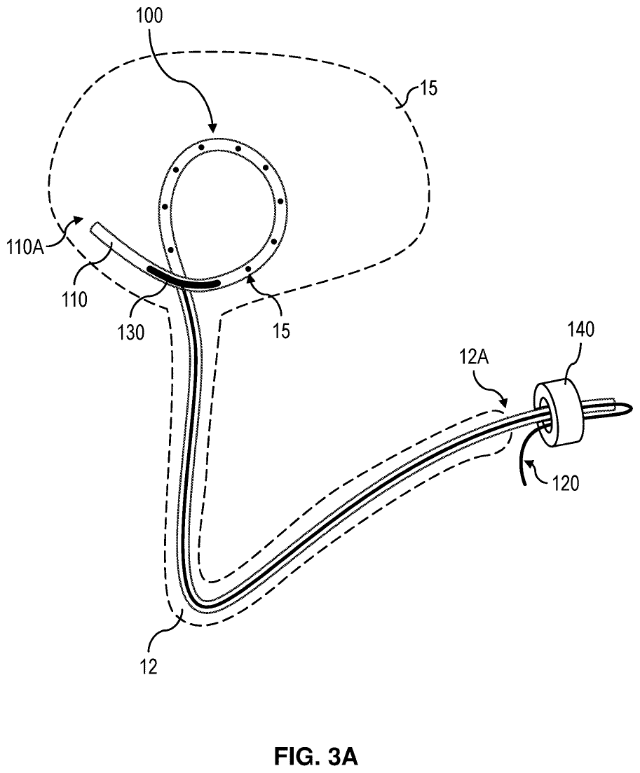

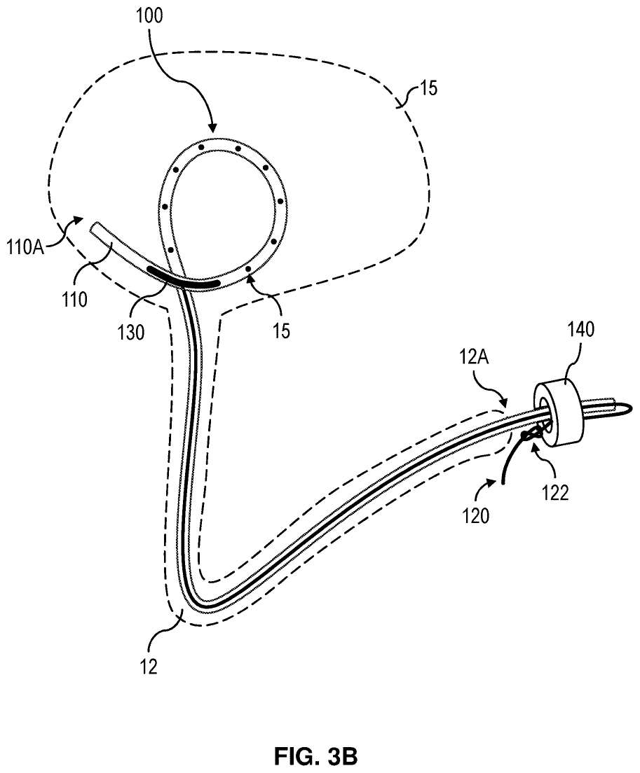

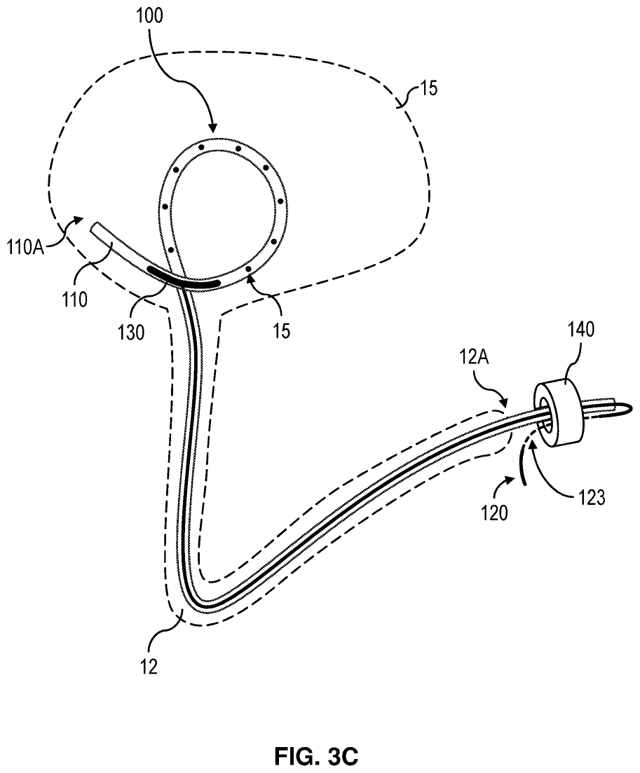

FIG. 3A shows an exemplary catheter system 100 with a portion of the tubular body 110 arranged in a patient's urethra 12. The catheter system 100 is shown in cross-section, except for the cuff 140 arranged near the distal end of the body. As shown, the system may be arranged in a looped configuration in a patient's bladder 15, with a stiffener 130 abutting against a bladder neck. The stiffener 130 may reinforce the looped tubular body and reduce the risk of the system unexpectedly falling through the urethra. In some embodiments, the tether 120 of the system may extend from a fixation point on the stiffener 130 out of a patient's orifice 12A. The tether 120 may be axially fixed to the tubular body through a cuff 140, to reduce the risk of the catheter system reverting back to the relaxed configuration. When the catheter system is no longer required in the patient's urethra, the clinician may remove the cuff from the tubular body (and/or otherwise separate the cuff from the body) and allow the catheter system to relax and be readily removable from the urethra 12 and bladder 15.

In some embodiments, the distal end of the tubular body may be in fluid communication with a fluid source and/or collection container through any suitable coupling means (e.g., a Luer-lock coupling) to allow fluid flow through the catheter systems. For example, the catheter system 100 may be used to drain urine from the bladder 15 into a diaper. Accordingly, urine may flow from the bladder 15 and into the tubular body 110 through one or more drainage holes 115, as described previously. The exemplary drainage holes 115 of the cross-sectional view of the body shown in FIG. 3A are shown on the back side of the body. As described previously, the drainage holes may be arranged in any suitable orientation relative to the axial direction of the tubular body, as the present disclosure is not so limited. The urine may then flow to the distal end of the tubular body and into a collection container. In some embodiments, fluid may flow into the bladder through the one or more drainage holes to help flush the urethra 12 and/or bladder 15. It should be appreciated that the catheter system may be coupled to any conventional component (e.g., fluid source, collection bag, etc.) through any suitable coupling, as the present disclosure is not so limited.

In some embodiments, the distal end of the tether 120 may include one or more features to help inform the clinician of how far the tether has been extended. The features can provide visual (e.g., through color or other visually distinct properties) and/or tactile feedback (e.g., through tension or resistance) regarding the extension of the tether and thereby the state of the catheter system within the patient, which would not be visible to the clinician during operation. The features may provide information regarding the tension state of the tether, which may be at an appropriate level, under-tightened (which may increase the risk of dislodgement if the catheter system is not properly looped), or overtightened (which may deform the tubular body into a telescoping configuration or may result in the formation of various smaller loops, which may reduce the rate of fluid flow into and/or out of the tubular body). In some embodiments, an overtightened tether may also increase the risks of the tether pulling through the inlet/outlet ports. The marker features at the distal end of the tether may better inform the clinician regarding the state of the tether tension and reduce the aforementioned risks associate with poor tensioning.

In some embodiments, the distal end of the tether 120 may include a knot 122, as shown in FIG. 3B. The knot may serve as a tactile stopper, providing resistance to withdrawal past the cuff 140, and limiting the risk of accidental release of the catheter system during use.

The knot 122 may be sized and shaped to pass between the cuff 140 and the tubular body 110, but may require an extra bit of effort, thereby providing tactile feedback to the clinician. It should be appreciated that although a knot is shown in FIG. 3B, alternative features, such as a knob on the tether, a thickened portion, a roughened portion, a portion with different texture relative to the rest of the tether and/or any other feature which may be palpably distinct, are also contemplated. It should also be appreciated that although a single knot is shown in FIG. 3B, more than one tactile marker is also contemplated.

In some embodiments, the distal end of the tether may include one or more visual markers to provide feedback regarding the extension of the tether, and thereby the state of the catheter system within the patient, which would not be visible to the clinician during operation. For example, as shown in FIG. 3C, the distal end of the tether 120 may include a portion 123 which may be visually distinct from the remainder of the tether 120. For example, in some embodiments, the portion 123 may be colored and/or may include other indicia to help visually indicate the extension of the tether to the clinician. The visualization of the portion 123 indicated in FIG. 3C may inform the clinician that the catheter system is in the desired looped configuration and at the appropriate level of tension. It should be appreciated that any number of colors in any suitable arrangement may be employed. In some embodiments, a gradient of colors may be used for the tether portion 123 to indicate a graduate change in tether tension. In other embodiments, the tether portion 123 may be a single color (or shade or hue) distinct from the remainder of the tether 120. In some embodiments, marker features from FIGS. 3B-3C may be combined to provide simultaneous visual and tactile feedback to the clinician for redundancy purposes.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

What is claimed is:

1. A catheter system comprising:
a tubular body, the tubular body having a proximal end configured to be arranged in a bladder of a patient;
a reinforced portion proximal to and offset from the proximal end of the tubular body; and
a tether coupled to the reinforced portion, the tether extending from the reinforced portion to a distal end of the tubular body, wherein at least a portion of the tether is disposed inside the tubular body.

2. The catheter system of claim 1, wherein the tether comprises a handling portion arranged proximate to and external from the distal end of the tubular body, and wherein the handling portion is configured to be grasped to tension the tether and transition the catheter system from a relaxed configuration to a looped configuration.

3. The catheter system of claim 1, wherein the reinforced portion is integral with the tubular body.

4. The catheter system of claim 1, wherein the tether is coupled to a central portion of the reinforced portion.

5. The catheter system of claim 1, wherein a central portion of the reinforced portion is spaced approximately 3 cm from the proximal end of the tubular body.

6. The catheter system of claim 1, wherein an axial length of the reinforced portion is approximately 3 cm.

7. The catheter system of claim 1, wherein the reinforced portion comprises a stiffener body disposed inside the tubular body.

8. The catheter system of claim 1, further comprising:
an outlet port formed on the tubular body at a first location; and
an inlet port formed on the tubular body at a second location, wherein the second location is arranged between the first location and the distal end of the tubular body.

9. The catheter system of claim 8, wherein the tether is configured to be disposed outside of the tubular body between the outlet port and the inlet port.

10. The catheter system of claim 8, wherein the outlet port is spaced approximately 9 cm from the inlet port.

11. The catheter system of claim 1, further comprising one or more drainage holes.

12. The catheter system of claim 8, further comprising one or more drainage holes axially arranged between the inlet port and the outlet port.

13. The catheter system of claim 1, further comprising a cuff configured to be coupled to the distal end of the tubular body.

14. The catheter system of claim 1, wherein the tether is a surgical suture.

15. The catheter system of claim 1, wherein the distal end of the tubular body is in fluid communication with a fluid container and/or a fluid source.

16. The catheter system of claim 1, wherein the proximal end is configured to be arranged in a bladder of a pediatric patient.

17. The catheter system of claim 1, further comprising one or more markers arranged proximate to a distal end of the tether.

18. The catheter system of claim 19, wherein the marker is at least one of a visual marker and a tactile marker.

19. A method of operating a catheter system, the method comprising:
grasping a distal end of a tether coupled to a reinforced portion of the system, the reinforced portion disposed proximate to a proximal end of a tubular body; and
transitioning the tubular body from a relaxed configuration to a looped configuration.

20. The method of claim 19, wherein applying grasping the distal end of the tether comprises applying tension to the tether.

21. The method of claim 19, wherein the tether is coupled to a central portion of the reinforced portion.

22. The method of claim 19, wherein the reinforced portion comprises a stiffener body disposed inside the tubular body.

23. The method of claim 19, wherein applying tension to the tether comprises:
sliding the tether through an outlet port formed on the tubular body at a first location; and
sliding the tether through an inlet port formed on the tubular body at a second location, wherein the second location is arranged between the first location and the distal end of the tubular body.

24. The method of claim 23, further comprising sliding the tether external to the tubular body between the outlet port and the inlet port.

25. The method of claim 19, further comprising locking axial displacement of the tether relative to the tubular body with a cuff, the cuff configured to be coupled to the distal end of the tubular body.

26. The method of claim 19, further comprising fluidically coupling the distal end of the tubular body with a fluid container and/or a fluid source.

27. The method of claim 19, further comprising draining fluid from a patient's bladder through one or more drainage holes.

28. The method of claim 19, further comprising flushing fluid into a patient's

29. The method of claim 20, further comprising sensing the tension of the tether through one or more markers arranged proximate to a distal end of the tether.
